(19) United States
(12) Patent Application Publication
(10) Pub. No.: US 2013/0331298 A1
(43) Pub. Date: Dec. 12, 2013

(54) ANALYZER AND DISPOSABLE CARTRIDGE FOR MOLECULAR IN VITRO DIAGNOSTICS

(71) Applicant: Great Basin Scientific, West Valley, UT (US)

(72) Inventor: Larry Rea, Salt Lake City, UT (US)

(21) Appl. No.: 13/911,878

(22) Filed: Jun. 6, 2013

Related U.S. Application Data
(60) Provisional application No. 61/656,380, filed on Jun. 6, 2012.

(51) Int. Cl. C12Q 1/68

(52) U.S. Cl. CPC C12Q 1/6844
USPC 506/16; 435/309.1

(57) ABSTRACT

An in vitro diagnostics analyzer and assay cartridge for carrying out biochemical assays is disclosed. The analyzer includes a tilted clamp assembly for holding an assay cartridge, upper and lower motor assemblies for manipulating the assay cartridge, and an optical reader. The cartridge includes an injection port for receiving a biological sample, a central channel through which the sample passes, one or more processing chambers, one or more reagent containers, a detection chamber, and optionally a waste chamber. The analyzer and cartridge may be used for detection of a variety of analytes, including pathogens for medical diagnostics.

### RELATED APPLICATIONS

The present patent document claims the benefit of the filing date under 35 U.S.C. §119(e) of Provisional U.S. Patent Application No. 61/656,380, filed Jun. 6, 2012, which is hereby incorporated by reference.

### BACKGROUND

The present invention relates to devices for use in diagnostic assays, and more particularly to analyzers and cartridges for carrying out biochemical assays, such as for immunoassays, nucleic acid extraction, amplification, and detection from clinical samples.

There has been a growing reliance on micro scale diagnostic assays instead of clinical, laboratory diagnostic assays from macro scale samples owing to specimen volume requirements decreasing from milliliters to microliters, and continuing reduction of assay times from days to hours even minutes. While these improvements are due in part to advances in materials and fabrication, the rapidity of mass and heat transfer at the micro scale and increasing detection sensitivity represent a continuing area for innovation. More can be accomplished to improve sensitivity, accelerate detection, and broaden assay platforms for investigating a variety of biological causes of disease.

For example, Clostridium difficile is an anaerobic, gram-positive, spore-forming bacterium. Infection by toxin-producing C. difficile causes a spectrum of disease symptoms from mild diarrhea to fulminant pseudomembranous colitis. Although C. difficile is apparently an ancient species, emerging more than 1 million years ago, it has been recognized as a human pathogen for only 3 decades, with dramatic increases in both hospital and community acquired infections in the past decade.

Diagnostic testing for toxigenic C. difficile has been traditionally accomplished by sensitive and specific, but time-consuming culture methods, as well as by immunoassays, which are faster but in general do not have sufficient sensitivity. Immunoassays that detect the GDH antigen display high sensitivity but poor specificity for C. difficile. Further, the GDH assays do not determine toxigenic status. This has led some laboratories to adopt 2-step algorithms in which samples that test positive using a GDH immunoassay are followed by a molecular assay to determine whether C. difficile is present and whether it is toxigenic. In comparison to such 2-step algorithms, molecular tests alone have increased sensitivity and specificity but are more costly.

There is, therefore a need to innovate automatic diagnostic assays for detecting biological causes of disease such as pathogen infections and be more cost-effective.

### SUMMARY

In one aspect, an assay cartridge is disclosed having an injection port for receiving a sample; a central channel originating at the injection port; a plurality of processing chambers connected to the central channel; a plurality of reagent containers connected to the central channel; and optionally a waste chamber. In some embodiments, the cartridge also includes at least one bubble trap for removing air from the sample, the bubble trap being in fluid communication with the central channel or a fluid channel. In some embodiments, the cartridge has a second bubble trap in fluid communication with at least one of the plurality of reagent containers.

In some embodiments, the plurality of chambers include a first mixing chamber for sample preparation; an amplification chamber for amplifying a target genomic DNA suspected of being present in the biological sample, the chamber further comprising lyophilized amplification enzymes; and a detection chamber having an array of probes immobilized on a silicon chip surface. In some embodiments, the amplification chamber contains lyophilized thermophilic helicase-dependent enzyme.

In some embodiments, the plurality of reagent containers include a first washing reagent container containing a washing medium; a conjugating reagent container containing a conjugating agent; and a precipitating reagent container containing precipitating reagent. In some embodiments, the plurality of reagent containers also includes a dilution reagent container containing a dilution medium and a hybridization reagent container containing a hybridizing reagent.

In some embodiments, the conjugating agent includes biotin-labeled primers complementary with some sequence within a variable region of a specific gene in the target genomic DNA. In some embodiments, the hybridizing reagent is anti-biotin antibody conjugated to the horseradish peroxidase enzyme. In some embodiments, the precipitating reagent is 3,3′,5,5′-tetramethylbenzidine.

In some embodiments, the assay cartridge is tilted such that the detection chamber is at a higher elevation than the injection port.

In some embodiments, the cartridge also includes a plurality of thermal pads located adjacent to one or more of the processing chambers that require heating. In some embodiments, the also has one or more stirring rods, each of which is located in a processing chamber for mixing reagents and a sample.

In another aspect, an in vitro diagnostics analyzer is disclosed having a tilted clamp assembly configured to hold an assay cartridge; upper and lower motor assemblies operably connectable to one or more control valves, lances, and blister packs; and an optical reader.

In some embodiments, the analyzer also includes optical sensors for monitoring fluid flow through the cartridge.

### BRIEF DESCRIPTION OF THE DRAWINGS

FIG. 1 depicts a cartridge layout and a related analyzer according to one embodiment of the invention.

FIG. 2 depicts the analyzer of FIG. 1 with the access bay door opened to show where a cartridge can be mounted.

FIG. 3 depicts the analyzer of FIG. 1 with a cartridge mounted in the access bay.

FIG. 4 depicts an elevational view of the top of a cartridge according to one embodiment of the invention.

FIG. 5 depicts the bottom view of the cartridge depicted in FIG. 4.

FIG. 6 depicts the analyzer of FIG. 1 showing internal components for manipulating a cartridge.

FIG. 7 depicts a bubble trap useful in embodiments of the invention.

FIG. 8 depicts components of a kit for conducting an assay using embodiments of the invention.

### DETAILED DESCRIPTION

Units, prefixes, and symbols may be denoted in their SI accepted form. The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

The following definitions are provided as an aid in interpreting the claims and specification herein. Where works are cited or incorporated by reference, and any definition contained therein is inconsistent with that supplied here, the definition used herein should be applied.

As used herein, the term “sample” or “samples” refers to samples taken from a patient. Such samples may be taken by, for example, gingival, buccal, mucosal epithelial, saliva, wound exudates, pus, surgical specimens, lung and other respiratory secretions, nasal secretions, sinus drainage, sputum, blood, urine, medical and inner ear contents, ocular secretions and mucosa, cyst contents, cerebral spinal fluid, stool, diarrheal fluid, tears, mammary secretions, ovarian contents, ascites fluid, mucous, gastric fluid, gastrointestinal contents, urethral discharge, vaginal discharge, vaginal mucosa, synovial fluid, peritoneal fluid, meconium, amniotic fluid, semen, penile discharge, and the like. Samples representative of mucosal secretions and epithelia are acceptable, for example mucosal swabs of the throat, tonsils, gingival, nasal passages, vagina, urethra, rectum, lower colon, and eyes. Besides physiological fluids, samples of water, industrial discharges, food products such as milk, air filtrates, and so forth can be specimens for samples.

As used herein, the terms “target analyte,” “target molecule,” and “analyte of interest” may include a nucleic acid, a protein, an antigen, an antibody, a carbohydrate, a cell component, a lipid, a receptor ligand, and so forth. The microfluidic analytical device disclosed herein is configured to detect a target molecule of these sorts singly or in combinations.

As used herein, the terms “cartridge” and “card” with fluidic structures and internal channels and chambers having microfluidic dimensions. These fluidic structures may include chambers, valves, vents, traps, inlets, outlets, windows, and containers, for example. Microfluidic cartridges may be fabricated from various materials using techniques such as laser stenciling, embossing, stamping, injection molding, masking, etching, and three-dimensional lithography. Laminated microfluidic cartridges are further fabricated with adhesive interlayers or by adhesiveless bonding techniques such as by thermal or pressure treatment of oriented polypropylene or by ultrasonic welding. The microarchitecture of laminated and molded microfluidic cartridges can differ according to the limitations of their fabrication methods and the design requirements.

As used herein, the term “blister pack” and “reagent container” refers to an on-board reagent pack or sachet under a deformable (or elastic) diaphragm. Blister packs can be used to deliver reagents by pressuring the diaphragm and may appose a “sharp,” such as a metal chevron, so that pressure on the diaphragm ruptures a pillow. Alternatively, the pillow may be pierced by a lance adjacent to the blister pack. The blister pack and reagent containers may be used with check valves or closable vents to produce directional fluid flow and reagent delivery. Elastic diaphragms are readily obtained from polyurethane, polysilicone and polybutadiene, and nitrile for example (see elastomer). Deformable, inelastic diaphragms are made with polyethylene terephthalate (PET), mylar, polypropylene, polycarbonate, or nylon, for example. Other suitable materials for the deformable film include parafilm, latex, foil, and polyethylene terephthalate. Factors considered for selecting a deformable film include material compatibility with the reagent to be stored and the yield point and the deformation relaxation coefficient (elastic modulus). Use of such reagent containers permits delivery or acceptance of a fluid while isolating the contents of the microfluidic device from the external environment, and protecting the user from exposure to the fluid contents.

As used herein, the term “single entry” refers to a microfluidic device, card or cartridge that requires, or permits, only a single introduction of sample, and the assay is then conducted within a self-contained, sealed system. Such devices optionally contain a device for sealing or locking the sample port and an on-board means for disinfecting the contents of the device and any waste following completion of the assay.

As used herein, the term “waste chamber” refers to a cavity or chamber that serves as a receptacle for sequestering discharged sample, rinse solution, and waste reagents. In some embodiments, the waste chamber may include a wicking material or wick. In some embodiments, the waste chamber may also be sealed under an elastic isolation membrane sealingly attached to the body of the microfluidic device. In some embodiments, this inner membrane expands as bibulous material inside it expands, thus enclosing the waste material. In some embodiments, the cavity outside the isolation membrane is vented to atmosphere so that the waste material is contained and isolated. In some embodiments, the waste chamber may include dried or liquid sterilants.

As used herein, the term “vent” refers to a pore intercommunicating between an internal cavity or microfluidic channel and the atmosphere. A “sanitary” or “isolation vent” refers to a vent having a filter element that is permeable to gas, but is hydrophobic or oleophobic and resists wetting. Optionally, these filter elements have pore diameters of 0.45 microns or less. In some embodiments, these filters function both in forward and reverse isolation. Filter elements of this type and construction may also be placed internally, for example to isolate a valve from a pneumatic manifold controlling it.

As used herein, the phrase “means for extracting” refers to various cited elements of a device, such as a solid substrate, filter, filter plug, bead bed, frit, or column, for capturing target nucleic acids from a biological sample. Extracting further comprises methods of solubilizing, and relates to the resuspension of cells and tissue from a sampling device such as a swab. Generally, extraction means include a mechanical pumping component that promotes physical resuspension by turbulent or near turbulent flow. Such flow may be reciprocating flow, and may be pulsatile at varying frequencies to achieve the desired resuspension in a reasonable interval of time. Extraction means also include use of detergent-based buffers, sulfhydryl-reducing agents, proteolytics, chaotropes, passivators, and other solubilizing means.

As used herein, “means for amplifying” refers to techniques for duplicating nucleic acid. Such techniques include polymerase chain reaction (PCR). Other amplification techniques include thermophilic helicase-dependent (tHDA) amplification, LAMP (loop-mediated isothermal amplification of DNA), reverse transcription polymerase chain reaction (RT-PCR), ligase chain reaction (LCR), transcription-based amplification systems (TAS), including nucleic acid sequence based amplification (NASBA), “Rolling Circle”, “RACE” and “one-sided PCR.” Embodiments disclosed herein for microfluidic PCR should be considered representative and exemplary of a general class of microfluidic devices capable of executing one or various amplification protocols.

As used herein, “means for detecting” refers to an apparatus for displaying an endpoint, i.e., the result of an assay, and may include a detection chamber, and a means for evaluation of a detection endpoint. Detection endpoints are evaluated by a machine equipped with a spectrophotometer, fluorometer, luminometer, photomultiplier tube, photodiode, nephlometer, photon counter, and the like. Magnifying lenses in the cover plate, optical filters, colored fluids and labeling may be used to improve detection and interpretation of assay results. Fluorescence quenching detection endpoints are also contemplated. A variety of substrate and product chromophores associated with enzyme-linked immunoassays are also well known in the art and provide a means for amplifying a detection signal so as to improve the sensitivity of the assay, for example “up-converting” fluorophores. Detection systems can be qualitative, quantitative or semi-quantitative.

As used herein, “means for isolation” can refer to impermeable cartridge bodies, gas permeable hydrophobic venting, bibulous padding in a waste chamber, disinfectant in waste chamber, elastomeric membrane separating a pneumatic actuator from a blister pack, a valve with elastomeric membrane actuated by suction pressure, suction pressure in said sample entry port, on-board reagent pack, reagent container, self-locking single-entry sample port, gasketed closure, and disposable external skin or skins. Isolation refers both to the protection of the user from potentially biohazardous specimens, and to the protection of the specimen from contamination by the user or by foreign environmental materials.

As used herein, “closure means” or “means for sealingly closing” include caps, lids, threaded closures, “ziplock” closures, ball valves, gasketed closures, gaskets, seals, snap caps of all sorts, bungs, corks, stoppers, lip seals, press seals, adhesive seals, waterproof seals, single-entry seals, tamper-proof seals, locking seals, track-slidable sealable covers, compression seals, one-way valves, spring-loaded valves, spring-loaded lids, septa, tee-valves, snap-locking closures in general, piston-valves, double-reed valves, diaphragm closures, hinged closures, folding closures, Luer lock closures, and the like.

Unless the context requires otherwise, throughout the specification and claims which follow, the word “comprise” and variations thereof, such as “comprises” and “comprising” are to be construed in an open, inclusive sense, that is, as “including, but not limited to.”

The terms “about” and “generally” are broadening expressions of inexactitude, describing a condition of being “approximately” or “almost” in the sense of “just about,” where variation would be insignificant, obvious, or of equivalent utility or function, and further indicating the existence of obvious minor exceptions to a norm, rule or limit.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In one aspect, an in vitro diagnostics analyzer and disposable cartridge enables the detection of one or more pathogens from a patient (e.g. stool, mucus, sputum, blood or tissue). The device is designed to be operated by low skill level personnel with easy sample preparation, loading of the analyzer and execution of the analyzer operation.

The analyzer consists of an electromechanical device and controller. The analyzer has a clamp assembly to position and hold a disposable assay cartridge, upper and lower motor assemblies to control valves, lances and blister pack fluid movement, stirrer action, and an optical reader for determination of the assay results.

The analyzer is designed to run multiple assay types such as for detection of Clostriduim difficile, fungal, Staph ID and TB assays, although it can be used for detection of a wide variety of analytes. The analyzer and disposable cartridge are a flexible platform capable of mixing and heating samples for extraction and mixing samples, metering, diluting and rationing solutions. The analyzer can perform assays with isothermal or cycling (PCR type) heat controls and can detect and read one or several analytes on the detection chip in a heatable detection chamber. In some embodiments, the analyzer can read 1-20 analytes on a detection chip. In some embodiments, the analyzer can read 1-40 analytes on a detection chip. In some embodiments, the analyzer can read 1-60 analytes on a detection chip. In some embodiments, the analyzer can read 1-80 analytes on a detection chip. In some embodiments, the analyzer can read 1-100 analytes on a detection chip.

Biotin-labeled primers direct amplification of specific nucleic acid sequences, for example, a variable region of a gene for a pathogen's identification. Following the tHDA process, biotin-labeled, amplified target DNA sequences are hybridized to an array of probes immobilized on a silicon chip surface, then incubated with anti-biotin antibody conjugated to the horseradish peroxidase enzyme (HRP) or other appropriate chip development. The unbound conjugate is removed by washing and tetramethylbenzidine (TMB) is added to produce a colored precipitate at the location of the probe/target sequence complex. The resulting signal is detected by analyzer system.

Turning to an embodiment depicted in the figures, a molecular in vitro diagnostics system is disclosed. The diagnostic system enables in vitro diagnostic testing for the detection of analytes. The system includes three primary components: an assay cartridge 100 in which samples can be loaded for analysis, an analyzer 10 which manipulates cartridge 100, and a control platform 5 (not shown) such as a computer. The control platform 5 and analyzer 10 are connectable by an electronic communications means 15 such as a USB cable, serial cable, or wireless adaptors (not shown).

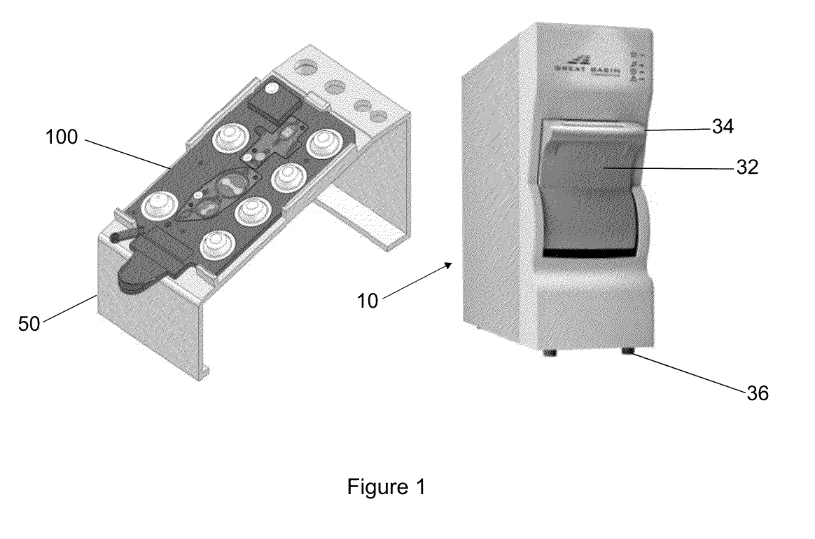

Now referring specifically to FIG. 1, analyzer 10 includes an access bay 30 and door 32 which may be opened by gripping handle 34. The analyzer may also be mounted on a plurality of rubber elastomeric feet 36.

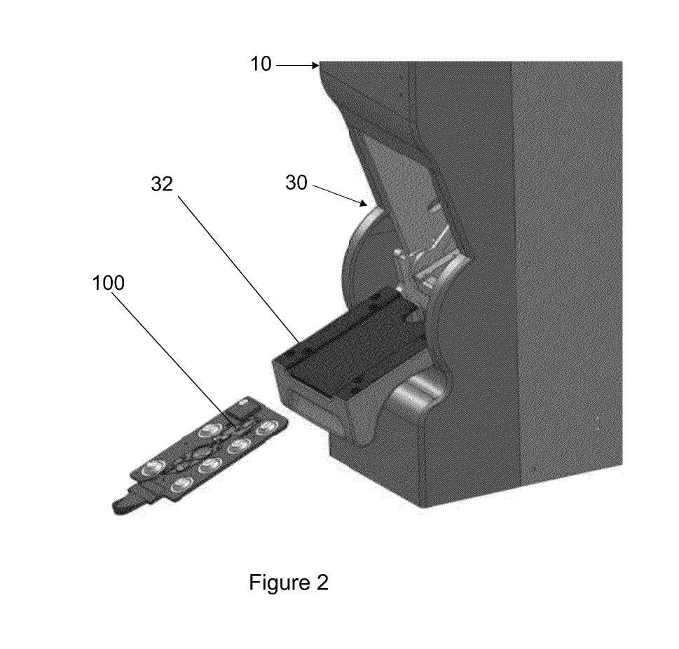

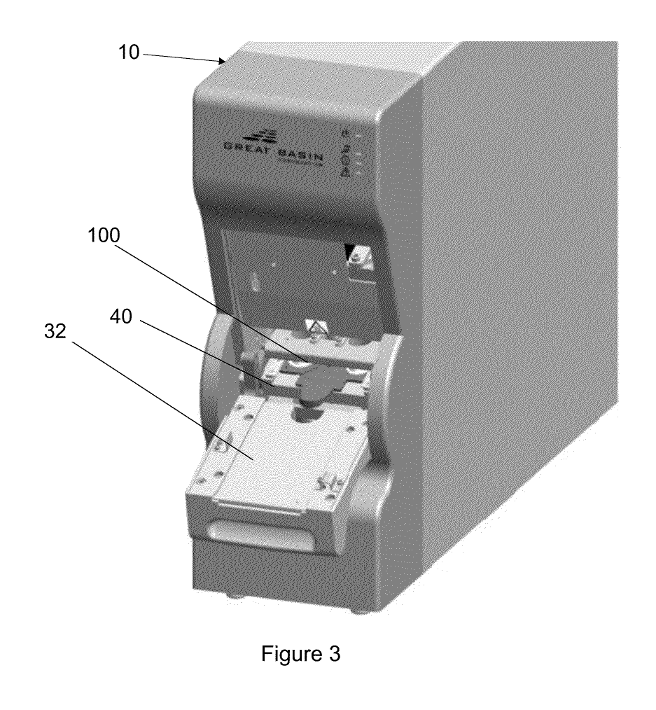

During sample preparation, cartridge 100 can be stored on jig 50. Referring to FIG. 2, access bay 30 may be accessed when door 32 is in an open configuration. Access bay 30 encloses cartridge platform 40 configured for receiving cartridge 100. Once loaded with a sample, cartridge 100 is placed into analyzer 10 on cartridge platform 40, for example as shown in FIG. 3. Platform 40 is part of a clamping assembly that holds cartridge 100 in a fixed position to other components found within analyzer 10.

In some embodiments, cartridge platform 40 is sloped or tilted at an angle so that it is not level with the surface on which analyzer 10 is mounted. The slope of the mounted cartridge is oriented such that an inlet or sample port is lower in elevation than a detection chamber as further described herein.

In some embodiments, cartridge 100 is a disposable cartridge capable of performing extraction, amplification, and detection steps in an enclosed system. The cartridge can have a variety of reagent containers, fluidic channels, processing chambers, and the assay chip coated with an array of sequence-specific detection probes. Reagents are contained within the integrated reagent containers. Amplification enzymes that are lyophilized can be stored in an amplification chamber.

A prepared sample is placed into a sample port of the cartridge for processing. Fluidic channels integral to the assay cartridge carry reagents from reagent containers to processing chambers where reagent mixing and sample processing occur. A waste chamber, self-contained and segregated within the test cartridge, receives and stores reagent waste.

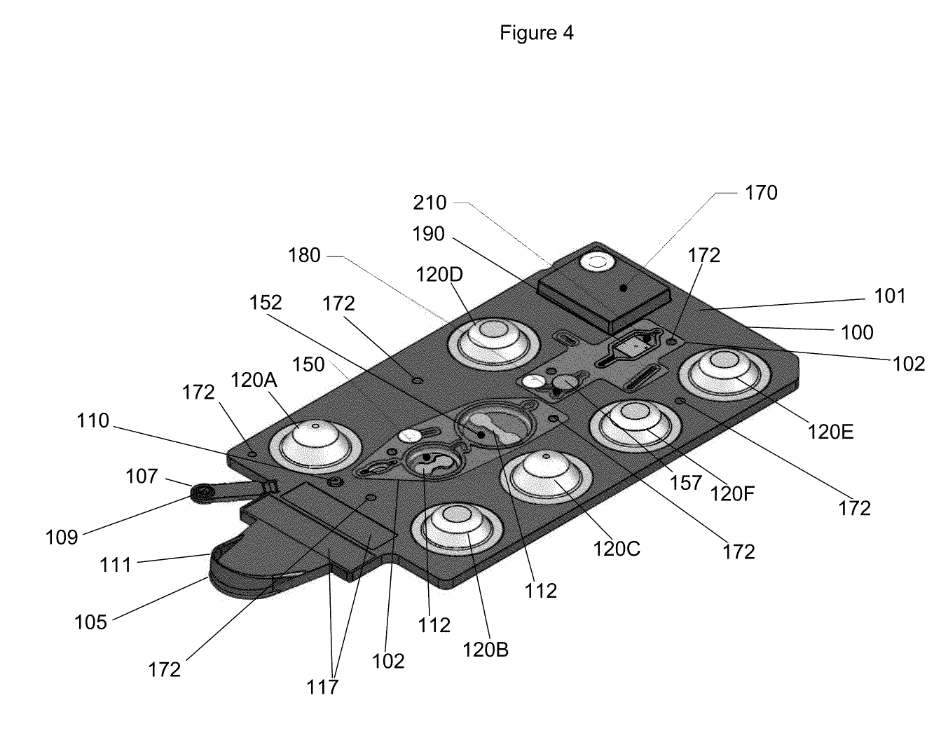

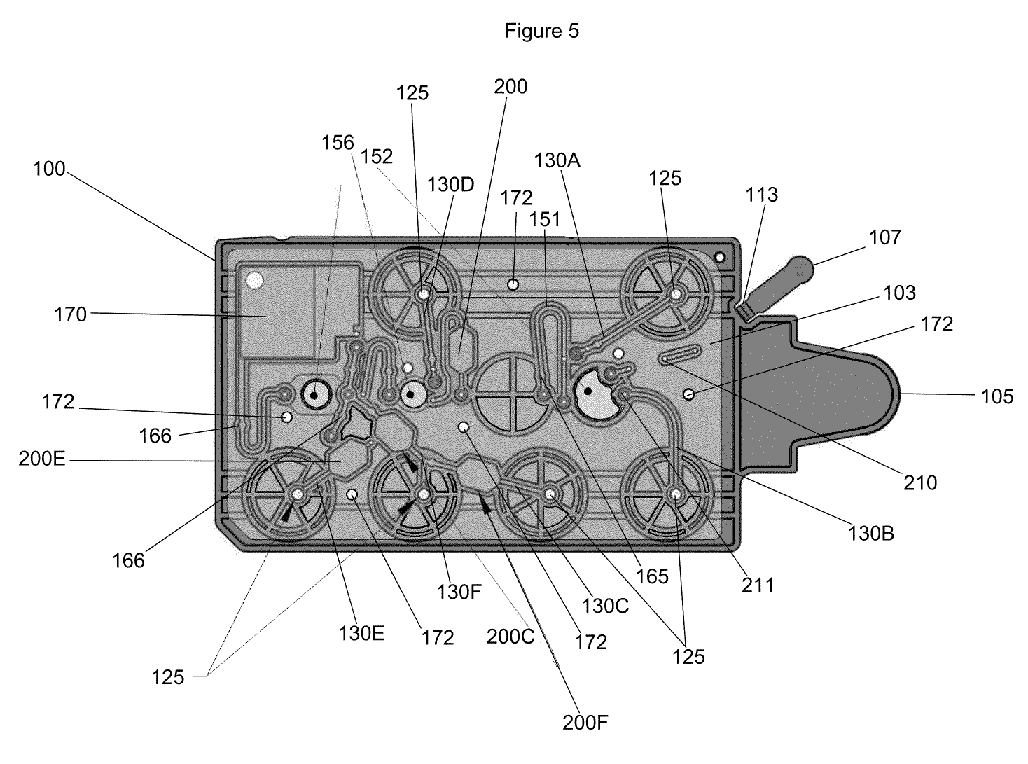

Referring to FIGS. 4 and 5, cartridge 100 is displayed in more detail. Cartridge 100 is made from an injection molded plastic base 101 having a plurality of ridges and compartments. The ridges and base, when covered by film 102 on one side of the base and film 103 on the opposite side of the base form enclosed microfluidic channels through which fluids may pass. The films also form a barrier for one or more chambers in which reactions occur biological samples are processed.

Base 101 may be formed of a polymeric material such as polypropylene. In some embodiments, the polymeric material is colored such as a polypropylene with 2% carbon black. The films may be formed of a polymeric material such as polypropylene. In some embodiments, the films are a transparent material made of polypropylene.

Films 102 and 103 may adhere to base 101 with an adhesive. In some embodiments, films 102 and 103 are thermally adhered to base 100 by heating.

Cartridge 100 can include a variety of components. For example, the cartridge may have an extended handling tab 105 which permits an operator to grip cartridge 100 without having to handle any of the other components. In some embodiments, the handling tab includes an extended lip 111 such as an in the embodiment shown in FIG. 4. The extended lip 111 can provide an additional surface for the operator to handle and grip.

Cartridge 100 includes a sample port (sometimes referred to as an inlet port) 110 through which a biological sample (not shown) can be inserted or injected into cartridge 100. A sample port closure tab 107 located near sample port 100 extends outward and in the same plane as cartridge 100. Closure tab 107 has a narrowed thickness 113 near a point where bending is desired so that after a sample is inserted into the sample port, closure tab 107 can be bent over and placed adjacent to sample port 110 thereby sealing off the port. In some embodiments, such as the embodiment shown in FIG. 4, closure tab 107 has two or more concentric circles 109 configured to fit into and around sample port 110. Alternatively, a separate lid (not shown) could be placed on sample port 110 to close it.

Cartridge 100 may optionally include labeling surfaces 117 on which sample labels or instructions can be placed such as with stickers or printing. In some embodiments, such as one depicted in FIG. 4, labeling surfaces 117 can be located near or adjacent to handling tab 105.

Cartridge 100 also includes a central channel or pathway 140 through which a sample (not shown) having a fluid front makes it way from sample port 110 through one or more processing chambers and to a detection chamber. Thus, the central channel is not a single, fluidic channel but comprises a series channels and chambers and other components through which the sample passes. It should also be understood that central channel 140 refers to a channel to which other components connect and does not necessarily imply that the channel is located in the center of the cartridge 100.

Central channel 140 extends from sample port 110 through one or more processing chambers. The central channel may also pass through one or more bubble traps, by one or more vents, an amplification chamber and a detection chamber as described in more detail hereafter. The central channel may also be connected to a waste chamber 170 such as shown in FIGS. 4 and 5 into which reagent waste can be transferred.

Cartridge 100 may contain one or more processing chambers. For example, in the embodiment depicted in FIG. 4, processing chamber 150 is used for sample preparation and is capable of heating to 100° C. and actively mixing the solution. A second processing chamber 152 is located downstream from processing chamber 150 by a metering channel 151 and is capable of mixing the sample prior to amplification and connected thereto through central channel 140. The metering channel 151 may be monitored by an optical sensor (not shown) at micro chamber 165. The sensor can be used by the analyzer to determine microfluidic fluid volumes passing through the micro chamber. Other micro chambers 166 can be located throughout the central channel 140 to continuously measure or examiner fluid flow and volumes.

An amplification chamber 180 is used to amplify target nucleic acid through amplification. The amplification chamber may be adjacent to one or more thermal pads 156 and 157. In some embodiments, a single thermal pad may be used at the amplification chamber. In other embodiments, two thermal pads may be used. The amplification chamber can include lyophilized amplification reagents such as HDA.

A detection chamber 190 is used to identify whether a target nucleic acid is present in the biological sample. The detection chamber can include a silicon chip with capture probes bonded thereto. Multiple assays can be run in the base cartridge by changing the HDA reagents, capture probes and reagent container contents.

Central channel 140 may also pass through one or more valves. For example, referring to FIGS. 4 and 5, valve 210 is located near the sample port 110. The valve closes when pressure is exerted through either film 102 or film 103 or both contacting the film with the valve. Valve 210 may be opened by removing the contact force applied to a film so that fluid can pass through the valve and further through central channel 140. Other valves can be located throughout cartridge 100 depending on whether such a location may be desired for stopping or regulating fluid passage.

Cartridge 100 also has one or more reagent containers 120 for delivering reagents. The reagent containers 120 may be made from sealed foil pouches thermally adhered to base 101 at various locations of cartridge 100 sometimes referred to as blister packs. Access holes (not shown) in the base 101 are located adjacent the bottom end of a reagent container. Each access hole includes a lance 125. The lance when pressed by an external force through film 103 pierces a corresponding reagent container mounted on the opposite side of the base 101. Once pierced, fluid and reagent can flow from the reagent container to pass over and around the lance and into a fluidic channel and toward central channel 140 at an appropriate, desired location. Reagent containers 120 are connected to central channel 140 through a plurality of fluidic channels 130.

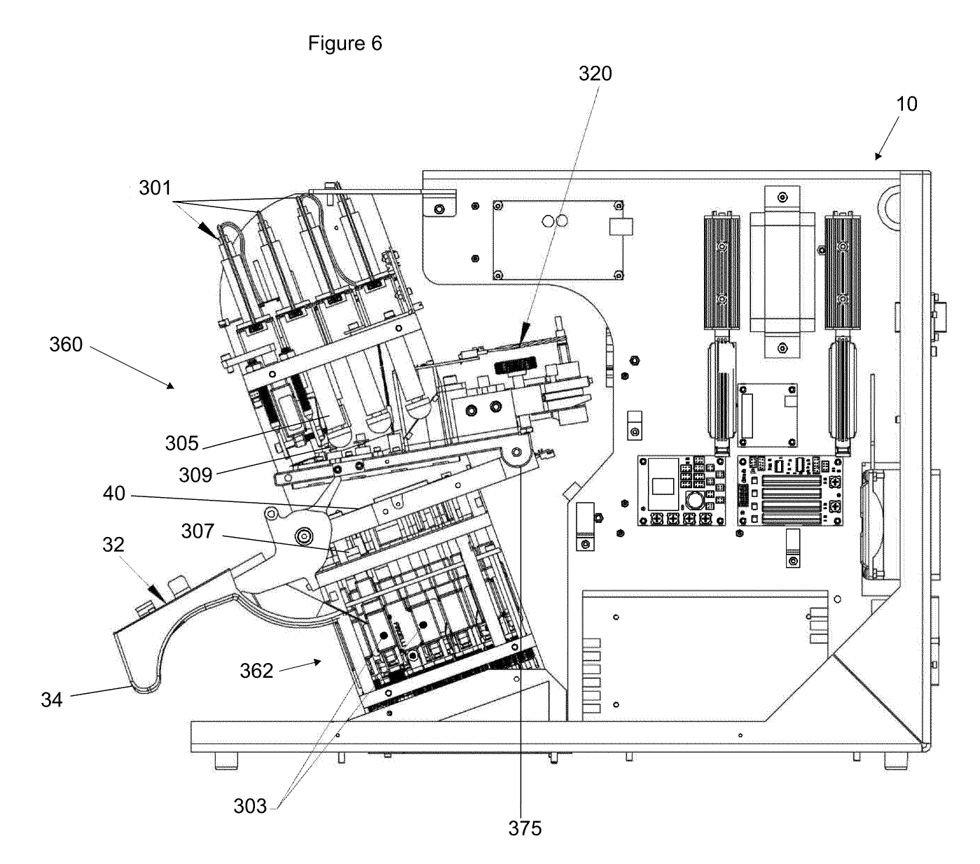

The reagent containers can be filled with a wide variety of appropriate reagents. For example, in the embodiment depicted in FIGS. 5 and 6, reagent container 120A can be filled with a dilution buffer. The dilution buffer can include a buffering agent such as Tris buffer. The dilution buffer may also include salts. The dilution buffer may also include surfactants. The dilution buffer may also include such as bovine serum albumin (BSA). The dilution buffer is used to obtain the correct ratio of a sample and salts for the proper amplification of the target analyte.

Another reagent container 120B can be filled with a sample extraction buffer.

Another reagent container 120C can be filled with a wash solution. The wash solution can include a buffering agent such as saline-sodium citrate (SSC). The wash solution can also include a surfactant. The wash solution can also include one or more preservatives. The wash solution is used to displace and wash a detection chip.

Another reagent container 120D can be filled with a hybridization buffer. The hybridization buffer can include a buffering agent such as saline-sodium citrate (SSC). The hybridization buffer can also include a surfactant. The hybridization buffer can also include one or more preservatives.

Another reagent container 120E can be filled with conjugate solution. The conjugate solution can include a buffering agent such as a sodium citrate buffer. The conjugate solution can also include salts. The conjugate solution can also include peroxidase conjugated monoclonal mouse antibody. The conjugate solution can also include one or more preservatives.

Another reagent container 120F can be filled with substrate solution. The substrate solution can include such as tetramethylbenzidine (TMB).

As already mentioned, the reagent containers are connected to central channel 140 through fluidic channels. For example, as shown in the embodiment depicted in FIGS. 5 and 6, reagent container 120B is connected to a first processing chamber 150 through fluidic channel 130B so that dilution buffer may be added to the sample. The fluidic channel may also include a valve 211 located at an opening into the processing chamber 150. The valve may be closed by pressure exerted on film 102 which seats the film against the valve 211 thereby blocking fluid from further travelling through fluidic channel 130B. When pressure is removed from 102 over valve 211, this permits fluid to pass through the valve and into processing chamber 150 when a lance has pierced the connected reagent container.

The reagent containers, when lanced, and the central channel 140 may also be in fluid communication with bubble traps. For example, bubble traps 200C, 200E, 200F are in fluidic channels 130C, 130E, and 130F respectively. Another bubble trap 208 is located in central channel 240 between processing chamber 152 and amplification chamber 180.

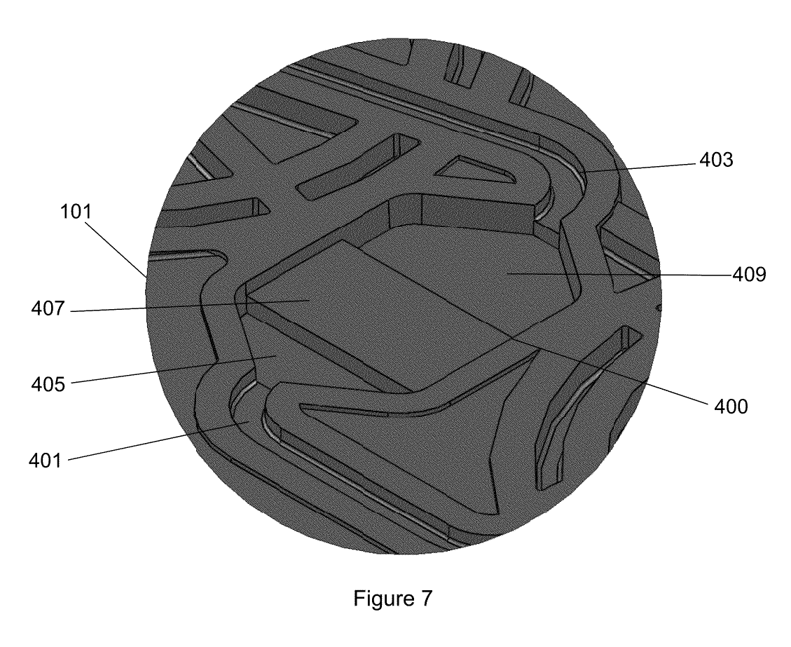

Referring to FIG. 7, an expanded view of a bubble trap 400 is shown. A fluidic channel 401 leads to an inlet space 405 of the bubble trap 400. A fluidic channel 403 leads away from an outlet space 409 of the bubble trap 400. Bubble trap 400 includes an inset 407 which has a height less than the height of the bubble trap inlet and outlet spaces 405 and 409. In some embodiments, the inset can be a separate component such as a silicon chip. Alternatively, in some embodiments, the inset can be a block intrinsically molded with the base 101.

Processing chamber 150 can also include a means for stirring or otherwise agitating the contents of the chamber with, for example, a magnetic stir bar 112. In some embodiments where heating of the processing chamber 150 is desired, such as one depicted in FIG. 5, the processing chamber can be adjacently located to thermal pad 154. When the thermal pad is activated, heat transfers to the fluid in the processing chamber thereby heating the sample and any reagents present in the chamber.

Central channel 140 goes from the first processing chamber 150 to a second processing chamber 152. The second processing chamber may also enclose means for stirring or otherwise agitating the contents of the chamber with, for example a magnetic stir bar 114. Reagent container 120A can also be connected to the second processing chamber by fluidic channel 130A so that extraction buffer may be added to the sample.

In some embodiments, it may be desirable to locate a vent in a central channel or other fluid channel where there can be a gas pocket (such as air) behind the fluid front of the sample. Such vents can be made of a hydrophobic material resistant to water contact through which the gas may pass and exit the cartridge 100. For example, in the embodiment shown in FIG. 4, vent 231 is located near central channel 140 and fluidic channel 120B. Vent 233 is also located near amplification chamber 180. A third vent 235 is located on waste chamber 170.

In some embodiments, the cartridge may have one or more reference holes 172. The reference holes may be used during manufacturing of the cartridge to hold for processing such application of the films 102 and 103, and mounting of reagent containers. Reference holes 72 may also be used for securing the cartridge to analyzer 10 and holding it in place while a diagnostic assay is carried out.

Depending upon the desired assay, the reagent containers can be arranged in different locations and filled with the same or similar reagents. In some embodiments, the number of reagent containers may be increased to provide for additional reagents. In some embodiments, the number of reagent containers may be decreased if reagents are unnecessary to the particular assay.

Referring to FIG. 6, an analyzer 10 is shown depicting internal components for manipulating a cartridge 100 (not shown). The internal components include a clamp assembly 375 which itself includes cartridge platform 40. Clamp assembly 375 secures cartridge 100 to upper clamp assembly 360 and lower clamp assembly 362. The upper and lower clamp assemblies 360 and 362 include a plurality of motors for controlling and manipulating reagents and fluid flow through the cartridge.

Upper clamp assembly 360 includes a plurality of stepper motors 301 operably connected to plungers 305. When activated, stepper motor 301 drives plunger 305 to contact and compress a corresponding reagent container 120. The compression of the reagent container forces fluid containing reagent from the reagent container into a corresponding fluidic channel. In some embodiments, plunger 305 includes a rounded end 306 (hemispherical, for example).

Lower clamp assembly 362 includes a plurality of two position linear motors 303 connected to pistons 307. In some embodiments, pistons 307 include a corresponding foot 308 which may have rounded ends 309. The linear motor 303, when activated, drives a corresponding piston 307 and corresponding foot 308 against the bottom side of cartridge 100. When piston 307 is oriented over a lance, 125 for example, then activating the linear motor can apply a force to the lance thereby piercing a corresponding reagent container 120. When piston 307 is oriented over a valve, 210 for example, a foot depresses film 102 against valve 210, thereby closing the valve and preventing fluid flow there through. Piston 307 can be withdrawn, thereby opening the valve and permitting fluid flow.

The clamp assembly can also include a plurality of optical sensors 380. The optical sensors can be mounted so that when cartridge 100 is loaded in analyzer 10, the sensors are adjacent processing chambers. The optical sensors can consist of a source and corresponding detector. When in operation, the source produces light from, for example, an LED. As a processing chamber fills, a film expands until it reaches a height that disrupts the detectors detection of the source light. The system can recognize such a disruption as an indication that the processing chamber is filled and stop further fluid flow by closing a valve or cease driving a motor that depresses a reagent container.

In some embodiments, a processing chamber can also be depressed by action of a stepper motor to drive fluid out the chamber and further through a central channel.

The internal components of analyzer 10 can also include a camera 320. Camera 320 can be located in analyzer 10 such that when cartridge 100 is loaded, camera 320 is over the detection chamber 190 to detect the presence or absence of target analytes.

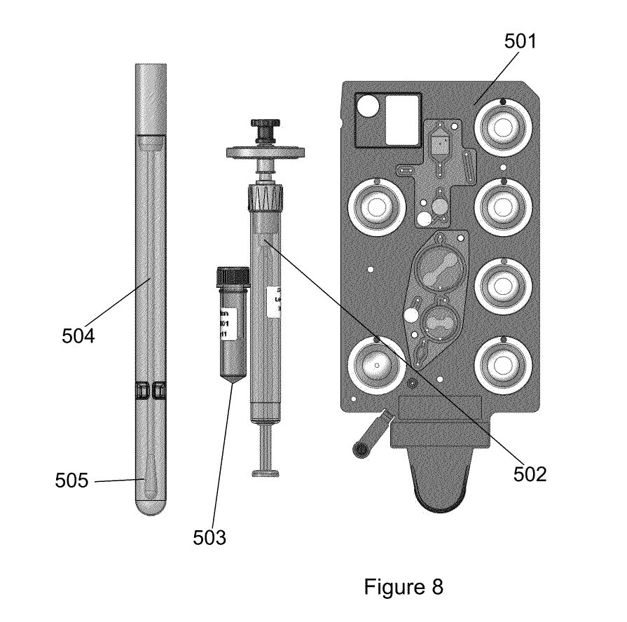

In another aspect, an assay kit is disclosed. The assay kit may include an assay cartridge 501, a spatula (not shown), a collection swab 504 in a container such as sterile container 505, a sample preparation syringe 502, and an extraction buffer tube 503 such as those depicted in FIG. 8.

A sample for pathology screening is obtained by first obtaining a sample, for example a stool sample from a patient. An extraction buffer is loaded into a sample preparation syringe device. A thoroughly mixed stool sample with spatula is swabbed, for example by covering the entire head of the swab in the sample. The swab tip with sample is can be broken off, for example at a pre-scored location, and immersed in the extraction buffer within the sample preparation device. The preparation device can then be vortexed for some period of time, twenty to thirty seconds for example. The vortexed sample and extraction buffer can then be passed through a filter and loaded into a sample syringe. The sample and extraction buffer can then be injected from the syringe into an inlet of the assay cartridge for analysis.

### EXAMPLES

1. C. difficile Assay

CDToxB was automated using an analyzer and disposable cartridge that performed the DNA extraction, amplification, and detection steps within an enclosed system. A disposable cartridge was manufactured by injection molding, and channels and fluid chambers are enclosed by welding of a clear-like plastic to the cartridge. A 6.7 mm² silicon chip with capture probes was bonded within a detection chamber. Reagent containers that store liquid reagents were attached, and lyophilized HDA reagents were added to the amplification chamber prior to welding on the cover. To perform a test, an operator swabbed the sample, vortexed and then filtered the swab in the sample preparation apparatus, and delivered 180 μL into the cartridge sample port. After closing the sample port, the cartridge was inserted into the analyzer, sample information is entered, and the test was initiated using a graphical user interface. The device, using a lance preposition on the cartridge, pierced the extraction reagent container and a plunger compressed the blister, expelling liquid into a mesofluidic (0.5 mm² cross-sectional area) channel. Optical sensors that detect fluid movement trigger blister motor and temperature control actions. Valves, controlled by 2-position linear actuator motors, are closed to isolate the chamber. Mixing was accomplished via a magnetic stir bar and the sample was heated via direct contact with a heater.

A second dilution was performed in the downstream control chamber, again with mixing, and the amplification chamber was filled thereby rehydrating lyophilized HDA reagents. For isothermal DNA amplification, this chamber was fluidically isolated and maintained 65±2° C. by intimate contact with a heat source. For detection, the amplified sample was diluted with hybridization buffer and introduced into a chamber where a 7 mm² silicon chip was affixed.

As for prior steps, fluidic movements and heater control performed the hybridization, washing, and signal development steps. The resulting eye-visible features were captured by a digital camera. Processing and filtering techniques minimize background and maintain the required signal-to-noise level. Multiple algorithms query pixel intensity and intensity gradient directionality to determine the presence or absence of a signal on each array feature. Once the optical reader software determined the presence or absence of signal on each array feature, a call logic tree was used to determine the assay result, which was displayed and reported automatically.

Clinical samples were tested with the BD GeneOhm CDiff PCR assay as the reference method, performed at a clinical site according to the manufacturer's recommendations (Becton Dickinson). In parallel, the sample was de-identified, blinded, and tested in singlet by automated CDToxB. Each sample was from a different patient. The lone discrepant result was from a heavily mucoid sample. Upon homogenization with a wooden spatula and repeat testing, the sample was CDToxB-positive. This sample was therefore resolved as positive and scored as false negative.

To calculate a limit of detection, logistic regression was used to fit a plot of CFU input versus the observed detection counts, and inverse prediction was used to find the predicted CFU value with a 95% probability of detection.

### Results

Automated Assay: Analytical Sensitivity, Specificity, and Testing of Clinical Samples.

An electromechanical instrument and disposable cartridge were developed and the automated assay was optimized to function equivalently to the manual assay in incubation times and temperatures. The disposable cartridge contains a port for sample introduction, control chambers for heating and mixing to extract DNA, an amplification chamber, and a detection chamber that houses the silicon chip. After loading the filtered sample, the assay is initiated using a graphical user interface. After 90 min, the CDToxB B test result is returned. Analytical sensitivity was addressed using dilutions of cultured C. difficile spiked into a pooled negative stool sample; at 20 CFU input, 20/20 tests were positive. At 10 CFU input 10/11 tests were positive, and at 4 CFU input, 6/19 tests were positive. Inverse prediction based on a logistic regression model fit to this data indicated that the automated CD-PaLoc detection limit is 10 CFU input to an amplification reaction (95% probability of detection). We then determined assay reactivity toward several C. difficile strains as well as toxigenic C. sordellii and non-clostridial species that can be present in stool samples. Each organism was spiked into a negative stool sample, and subsequent chip readouts indicated that all toxigenic C. difficile strains were detected, while toxigenic C. sordellii, non-toxigenic C. difficile and non-clostridial species tested negative (Table 1). Finally, to determine the ability to detect toxigenic C. difficile in clinical samples, 130 samples were tested alongside an FDA-approved PCR test. Discrepancies were resolved by toxigenic culture. Of these samples, one false negative was detected among the 32 positive samples and no false positives were observed, yielding 97% sensitivity (95% C.I. 82-99) and 100% specificity (95% C.I. 95-100). These initial experiments demonstrated automated assay function, paving the way for larger scale prospective clinical studies.

To combine the advantages of molecular testing (sensitivity) and immunoassays (low cost) we developed an assay for toxigenic C. difficile that couples isothermal DNA amplification to array-based hybridization. In lieu of monitoring nucleic acid amplification in real time, this approach permits inexpensive detection, requiring only a digital image instead of fluorophore-based detection with accompanying sophisticated optics and algorithms. Multiplexing is accomplished at two levels: at the amplification step and via hybridization to capture probes immobilized on the array. These methods were sufficient for detection of less than 10 C. difficile CFU in the context of a fecal sample. The ability of HDA to amplify crude fecal samples is also seen with other crude samples, for example blood culture. Straightforward filtration and automated dilution produces a simple test in which a swab sample is filtered and transferred into the cartridge to initiate testing.

PCR instruments used for moderately complex molecular diagnoses use microfluidics that require high manufacturing precision, precise temperature control for thermal cycling, and sophisticated optics for fluorescence detection. These requirements constrain instrument and test costs. In contrast, the analyzer/cartridge described here provides meso-scale fluidic movement, isothermal amplification, and eye-visible detection. Mesofluidic channels enable injection molding of a single plastic part. The isothermal DNA amplification is tolerant to variations of at least ±2° C., obviating the need for precise and rapid temperature changes that occur, perforce, in the PCR. By use of large visible features, the detection system can employ a digital camera rather than an expensive CCD imager. Taken together, mesofluidic design, isothermal DNA amplification, and eye-visible detection enable use of off-the-shelf components for analyzer construction, driving down instrument complexity and cost while maintaining ease of use. A limitation to the current automated test is the turnaround time of 90 minutes after test initiation, while the manual assay is performed in 60 min. The additional time is taken up by motor movements and mechanical calibrations; these factors have since been minimized to produce a 75 minute test.

The automated CDToxB assay described here, employing bpHDA and a minimal dilute-heat sample preparation procedure, has an LoD of 10 CFU input (at 95% detection confidence), while the manual assay could reliably detect as low as 1 CFU input. Thus the bpHDA method is comparable in sensitivity to other PaLoc amplification methods, many of which require DNA purification prior to amplification. These experiments demonstrated that the manually developed assay was successfully automated. Initial assessment using clinical samples portrays an accurate test and large studies are now required to establish clinical sensitivity and specificity.

1. An assay cartridge comprising:
an injection port for receiving a sample;
a central channel originating at the injection port;
a plurality of processing chambers connected to the central channel;
a plurality of reagent containers connected to the central channel; and
a waste chamber.

2. The assay cartridge of claim 1, further comprising at least one bubble trap for removing air from the sample, the bubble trap being in fluid communication with the central channel or a fluid channel.

3. The assay cartridge of claim 2, further a second bubble trap in fluid communication with at least one of the plurality of reagent containers.

4. The assay cartridge of claim 1, wherein the plurality of chambers include:
a first mixing chamber for sample preparation;
an amplification chamber for amplifying a target genomic DNA suspected of being present in the biological sample, the chamber further comprising lyophilized amplification enzymes; and
a detection chamber having an array of probes immobilized on a silicon chip surface.

5. The assay cartridge of to claim 4, wherein the amplification chamber contains lyophilized thermophilic helicase-dependent enzyme.

6. The assay cartridge of claim 1, wherein the plurality of reagent containers include:
a first washing reagent container containing a washing medium;
a conjugating reagent container containing a conjugating agent; and
a precipitating reagent container containing precipitating reagent.

7. The assay cartridge of claim 6, wherein the plurality of reagent containers also include:
a dilution reagent container containing a dilution medium; and
a hybridization reagent container containing a hybridizing reagent.

8. The assay cartridge of claim 6, wherein the conjugating agent includes biotin-labeled primers complementary with some sequence within a variable region of a specific gene in the target genomic DNA.

9. The assay cartridge of claim 7, wherein the hybridizing reagent is anti-biotin antibody conjugated to the horseradish peroxidase enzyme.

10. The assay cartridge of claim 6, wherein the precipitating reagent is 3,3′,5,5′-tetramethylbenzidine.

11. The assay cartridge of claim 4, wherein the assay cartridge is tilted such that the detection chamber is at a higher elevation than the injection port.

12. The assay cartridge of claim 1, further comprising a plurality of thermal pads located adjacent to one or more of the processing chambers that require heating.

13. The assay cartridge of claim 1, further comprising one or more stirring rods, each of which is located in a processing chamber for mixing reagents and a sample.

14. An in vitro diagnostics analyzer comprising:
a tilted clamp assembly configured to hold an assay cartridge;
upper and lower motor assemblies operably connectable to one or more control valves, lances, and blister packs;
an optical reader.

15. The analyzer of claim 14 further comprising a plurality of optical sensors for measuring fluid flow through the cartridge.
